(19) United States
(12) Patent Application Publication
(10) Pub. No.: US 2017/0130254 A1
(43) Pub. Date: May 11, 2017

(54) NUCLEOTIDE PRODUCTION PROCESS

(71) Applicant: NANJING UNIVERSITY OF TECHNOLOGY, Jiangsu (CN)

(72) Inventors: Hanjie YING, Nanjing City (CN); Zhi CAO, Nanjing City (CN); Yong CHEN, Nanjing City (CN); Xiaochun CHEN, Nanjing City (CN); Jinglan WU, Nanjing City (CN); Jingjing XIE, Nanjing City (CN)

(73) Assignee: Nanjing University of Technology, Jiangsu (CN)

(21) Appl. No.: 15/127,373

(22) PCT Filed: Mar. 26, 2014
(86) PCT No.: PCT/CN2014/074109

§ 371 Date: Nov. 22, 2016

(51) Int. Cl. C12P 19/30

(52) U.S. Cl. CPC C12P 19/30

(57) ABSTRACT

A nucleotide production process comprises: decomposing an RNA by using a nuclease P1 so as to obtain nucleotides AMP, GMP, CMP and UMP, converting part or all of the nucleotide AMP into a nucleotide IMP by using adenosine deaminase, separating the obtained nucleotide by using an ion exchange resin, and then performing concentration and crystallization to obtain purified nucleotides AMP, GMP, CMP, UMP and IMP or obtain purified nucleotides GMP, CMP, UMP and IMP. The whole biocatalysis production of nucleotides is implemented by using a double-enzyme catalysis method, and high-purity nucleotides are obtained by using an ion resin separation technology and a solvent crystallization method; and the production process is simple and environmentally-friendly, and has low costs, high product safety and purity.

### TECHNICAL FIELD

The present invention belongs to the technical field of biosynthesis and separation, in particular relates to a nucleotide production process.

### BACKGROUND ART

Nucleotide is a compound consisting of three materials comprising a base (purine base or pyrimidine base), a ribose (ribose or deoxyribose) and a phosphoric acid, which is the structural unit of ribonucleic acid (RNA) or deoxyribonucleic acid (DNA). Depending on the kinds of bases, nucleotides can be divided into adenylate (AMP), guanosine monophosphate (GMP), cytidine monophosphate (CMP), uridine monophosphate (UMP), thymidylate (TMP) and inosine monophosphate (IMP) and the like. Nucleotide compounds have important biological functions, they involve almost all biochemical reactions in vivo, including the following aspects: (1) existing as the structural units of biomolecules ribonucleic acid (RNA) and deoxyribonucleic acid (DNA); (2) adenosine triphosphate (ATP) in the cell acts as an energy currency, and uridine triphosphate (UTP) involves in the synthesis of glycogen for supplying energy; (3) adenylatethe is a component of coenzyme I, coenzyme II, flavin adenine dinucleotide (FAD) and coenzyme A (CoA), which involves various metabolic reactions in the body; (4) cyclic adenosine monophosphate (cAMP) and cyclic guanosine monophosphate (cGMP) are known as second messengers, and have certain regulation function on many basic biological processes; (5) uridine diphosphate (UDP) has a function of carrying and transfering glucose.

Nucleotide has an extremely important role in metabolic regulation of the body, and has very important nutritional value at the same time. From the beginning of the last century, nucleotide is used as a food additive well-liked by consumers. To improve the infant's immune function and memory, traces of nucleotides are added in the infant milk powder produced in Europe, America, Japan and other countries, and there are examples of adding RNA. In 1991, the European Community stipulated the supplement levels of nucleotides in the infant food, which is: in each 420 kg foods, CMP is 2.5 mg, UMP is 1.75 mg, AMP is 1.5 mg, GMP is 0.5 mg and IMP is 1.0 mg respectively. Announcement No. 15 of 2005 issued by the Minister of Health of the People's Republic of China recommended that the amount of nucleotides added in the infant formula milk powder is 0.2-0.58 g/kg (based on the total amount of nucleotides). Some Chinese patents introduce a high-energy milk which are added with nucleic acid or nucleotides and can be easily absorbed by the body and have functions of promoting blood circulation, improving brain function, promoting metabolism, anti-fatigue, anti-radiation, enhancing physical fitness, boosting immunity and so on. Nucleotides also have a function of strengthening freshness, so these kinds of nucleotides are called as flavor nucleotides. Especially guanosine monophosphate and inosine monophosphate (IMP+GMP) are known as strengthened MSG, the combination of the two nucleotides can make the effect on the flavor of MSG (monosodium glutamate (MSG)) increase dozens of times or even a hundred times. However, it is reported that the replacement of 2.5% IMP, 2.5% GMP and 95% MSG (4.7-7 kg) for 45 kg MSG has no significant effect on the product's flavor.

At present, the methods for producing nucleotides primarily comprise chemical synthesis method, natural raw materials extracting method, microbiological fermentation method, fermentation-catalyzed coupling method and double-enzyme catalysis method and so on. However, as there exsit some chemical toxic agents and many chemical byproducts, which are difficult to be completely removed from products, as well as some factors such as high requirements on equipments which are inappropriate to industrial productivity, the chemical synthesis method has been eliminated; the natural raw materials extracting method is difficult to meet the needs of the society due to limited sources of raw materials and being unable to realize a large mass production; it is feasible to use cheap raw material to produce nucleotides if the microbiological fermentation method is employed, however, this method is restricted as the ability of accumulating nucleotides for microbes is limited, the products are different to across through the cell membrane and the industrialisation is restricted due to high equipment assets, low concentration of products and high separation costs; the fermentation-catalyzed coupling method is a modified method aiming to overcome the shortcoming that nucleotides are different to across through the cell membrane, in this method, nucleosides were firstly produced by fermentation method, and then were phosphorylated to nucleotides. However, as there exsit byproducts such as 2′-phosphate, 3′-phosphate or diphosphate during the process of phosphorylation, which making separation difficult.

Patent application (CN1086219A) discloses a method for preparing nucleotides by chemically phosphorylating nucleosides, in which lower alkyl esters of phosphoric acid are used as the solvent and trihalo-phosphorus oxides are used as phosphorylating agents, however, there appear a large number of byproducts during the production process, which do not meet the requirements for food safety, meanwhile, acetic acid/ammonium acetate buffer is used during the elution process of resins, which bring difficulties for crystallisation.

Patent Application (CN1406273A) discloses a method for producing inosine acids (inosine monophosphates) by microbiological fermentation, compared with the traditional method, the accumulation amount of products has been significantly improved, but there still exists some shortcomings as it is difficult to be extracted.

In summary, the existing methods for producing nucleotides have some defects such as complexed production process, higher cost, being hard to separate, lower safety and purity, and being difficult to realize the industrial production and so on. The nucleotides, as biological products, have high nutrition and high medicinal value, which are commonly used for infant food and pharmaceutical industries, but the requirements of high purity and high quality make the existing process for producing nucleotides be difficult to meet the demands of infant food and pharmaceutical industries. Therefore, how to develop a new process for producing nucleotides, which is simple, and has low cost, high safety and high purity, to meet the demands of infant food and pharmaceutical industries has become a subject to be solved urgently in this field.

### SUMMARY OF THE INVENTION

To solve the above problem, the main object of the present invention is to provide a nucleotide production process, this process achieves the whole biocatalysis of nucleotides by using a double-enzyme catalysis method, and high-purity nucleotides are obtained by combining ion exchange resin separation technology and solvent crystallization method. The process is simple, environmentally-friendly and has low costs, by which the obtained product is safety and high purity.

To achieve the above object, the present invention provides a process for the production of nucleotides comprising: decomposing RNA by using nuclease P1 to obtain nucleotides AMP, GMP, CMP and UMP, converting part or all of the nucleotide AMP into nucleotide IMP by using adenosine deaminase enzyme, separating the obtained nucleotides by using an ion exchange resin, and then concentrating and crystallizing to obtain purified nucleotides AMP, GMP, CMP, UMP and IMP or obtain purified nucleotides GMP, CMP, UMP and IMP.

Preferably, the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in the same or different reactors.

Preferably, the reaction of decomposing RNA by using nuclease P1 to obtain nucleotides AMP, GMP, CMP and UMP and the reaction of converting part or all of the nucleotide AMP into nucleotide IMP by using adenosine deaminase are carried out in the same reactor, the process comprises the following steps:

step a1: in a reactor, decomposing RNA by using nuclease P1 to obtain nucleotides AMP, GMP, CMP and UMP, converting part or all of the nucleotide AMP into nucleotide IMP by using adenosine deaminase enzyme to obtain a mixture solution of nucleotides comprising AMP, GMP, CMP, IMP and UMP or a mixture solution of nucleotides comprising GMP, CMP, IMP and UMP,

step b1: separating the mixture solution of nucleotides obtained in step a1 by using an ion exchange resin, and then oncentrating and crystallizing to obtain purified nucleotides AMP, GMP, CMP, UMP and IMP or obtain purified nucleotides GMP, CMP, UMP and IMP;

preferably, the process comprises the following steps:

step a1: adding nuclease P1 and adenosine deaminase enzyme into a reactor, decomposing RNA to obtain nucleotides AMP, GMP, CMP and UMP, converting all of the nucleotide AMP into nucleotide IMP to obtain a mixture solution of nucleotides comprising GMP, CMP, IMP and UMP,

step b1: separating the mixture solution of nucleotides obtained in step a1 by using an ion exchange resin to obtain an IMP solution, a mixture solution containing UMP and IMP, as well as a mixture solution containing GMP and CMP respectively, separating the mixture solution containing UMP and IMP, as well as the mixture solution containing GMP and CMP respectively by using an anion exchange resin to obtain an UMP solution, an IMP solution, a GMP solution and a CMP solution, and then concentrating and crystallizing the UMP solution, the IMP solution, the GMP solution and the CMP solution respectively to obtain purified nucleotides UMP, IMP, GMP and CMP,

preferably, in step b1, letting the mixture solution of nucleotides obtained in step a1 flow through a cation exchange resin column, collecting the effluent to obtain a mixture solution containing UMP and IMP; eluting the cation exchange resin column adsorbed to reach a saturated state with deionized water, fractionally collecting the eluent to obtain an IMP solution and a mixture solution containing GMP and CMP; letting the mixture solution containing GMP and CMP as well as the mixture solution containing UMP and IMP flow through an anion exchange resin column, and eluting the anion exchange resin column adsorbed to reach a saturated state with deionized water, fractionally collecting the eluent to obtain an UMP solution, an IMP solution, a GMP solution and a CMP solution, and then concentrating and crystallizing the UMP solution, the IMP solution, the GMP solution and the CMP solution respectively to obtain purified nucleotides UMP, IMP, GMP and CMP;

preferably, the process comprises the following steps:

step a1: adding nuclease P1 and adenosine deaminase enzyme into a reactor, decomposing RNA to obtain nucleotides AMP, GMP, CMP and UMP, converting part of the nucleotide AMP into nucleotide IMP to obtain a mixture solution of nucleotides comprising AMP, GMP, CMP, IMP and UMP,

step b1: separating the mixture solution of nucleotides obtained in step a1 by using a cation exchange resin to obtain an AMP solution, an IMP solution, a mixture solution containing UMP and IMP and a mixture solution containing GMP and CMP respectively, separating the mixture solution of nucleotides containing UMP and IMP, as well as the mixture solution containing GMP and CMP respectively by using an anion exchange resin to obtain an UMP solution, an IMP solution, a GMP solution and a CMP solution, and then concentrating and crystallizing the AMP solution, the UMP solution, the IMP solution, the GMP solution and the CMP solution respectively to obtain purified nucleotides AMP, UMP, IMP, GMP and CMP,

preferably, in step b1, letting the mixture solution of nucleotides obtained in step a1 flow through a cation exchange resin column, collecting the effluent to obtain a mixture solution containing UMP and IMP; eluting the cation exchange resin column adsorbed to reach a saturated state with deionized water, fractionally collecting the eluent to obtain an AMP solution, an IMP solution and a mixture solution containing GMP and CMP; letting the mixture solution. containing GMP and CMP and the mixture solution containing UMP and IMP flow through an anion exchange resin column, and eluting the anion exchange resin column adsorbed to reach a saturated state with deionized water, fractionally collecting the eluent to obtain an UMP solution, an IMP solution, a GMP solution and a CMP solution, and then concentrating and crystallizing the AMP solution, the UMP solution, the IMP solution, the GMP solution and the CMP solution to obtain purified nucleotides AMP, UMP, IMP, GMP and CMP.

preferably, the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in different reactors, the process comprises the following steps:

step a2: in Reactor No. 1, decomposing RNA by using nuclease P1 to obtain a mixture solution of nucleotides containing AMP, GMP, CMP and UMP,

step b2: transferring the mixture solution of nucleotides obtained in the step a2 to Reactor No. 2, converting AMP in the mixture solution of nucleotides into IMP by using adenosine deaminase enzyme to obtain a mixture solution of nucleotides containing IMP,

step c2: separating the mixture solution of nucleotides containing IMP obtained in step b2 by using an ion exchange resin, and then concentrating and crystallizing to obtain purified nucleotide;

preferably, the process comprises the following steps:

step a2: in Reactor No. 1, decomposing RNA by using nuclease P1 to obtain a mixture solution of nucleotides containing AMP, GMP, CMP and UMP,

Step b2: transferring the mixture solution of nucleotides obtained in the step a2 to Reactor No. 2, converting all of AMP in the mixture solution of nucleotides into IMP by using adenosine deaminase enzyme to obtain a mixture solution of nucleotides containing IMP,

step c2: separating the mixture solution of nucleotides containing IMP obtained in step b2 by using a cation exchange resin to obtain an IMP solution, a mixture solution containing UMP and IMP, as well as a mixture solution containing GMP and CMP respectively, separating the mixture solution containing UMP and IMFP as well as the mixture solution containing GMP and CMP respectively by using an anion exchange resin to obtain an UMP solution, an IMP solution, a GMP solution and a CMP solution, and then concentrating and crystallizing the UMP solution, the IMP solution, the GMP solution and the CMP solution to obtain purified nucleotides UMP, IMP, GMP and CMP,

preferably, in step c2, letting the mixture solution of nucleotides containing IMP obtained in step b2 flow through the cation exchange resin column, collecting the effluent to obtain a mixture solution containing UMP and IMP; eluting the cation exchange resin column adsorbed to reach a saturated state with deionized water, fractionally collecting the eluent to obtain an IMP solution, and a mixture solution containing GMP and CMP; letting the mixture solution containing GMP and CMP and the mixture solution containing UMP and IMP flow through the anion exchange resin column respectively, and eluting the anion exchange resin column adsorbed to reach a saturated state with deionized water, fractionally collecting the eluent to obtain an UMP solution, an IMP solution, a GMP solution and a CMP solution, and then concentrating and crystallizing the UMP solution, the IMP solution, the GMP solution and the CMP solution respectively to obtain purified nucleotides UMP, IMP, GMP and CMP.

Preferably, the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in different reactors, the process comprises the following steps:

step a3: in Reactor No. 1′, decomposing RNA by using nuclease P1 to obtain a mixture solution of nucleotides containing AMP, GMP, CMP and UMP,

step b3: separating the mixture solution of nucleotides obtained in step a3 by using a cation exchange resin to obtain an AMP solution, an UMP solution, and a mixture solution containing GMP and CMIP respectively, separating the mixture solution containing GMP and CMP by using an anion exchange resin to obtain a GIMP solution and a CMP solution, and in Reactor No. 2′, converting all of the AMP solution into IMP solution by using adenosine deaminase enzyme,

preferably, in step b3, letting the mixture solution of nucleotides obtained in step a3 flow through a cation exchange resin column, collecting the effluent to obtain an UMP solution; eluting the cation exchange resin column adsorbed to reach a saturated state with deionized water, fractionally collecting the eluent to obtain a mixture solution containing GMP and CMP and an AMP solution; letting the mixture solution containing GMP and CMP flow through an anion exchange resin column, and eluting the anion exchange resin column adsorbed to reach a saturated state with deionized water, fractionally collecting the eluent to obtain a GIMP solution and a CMP solution, and in Reactor No. 2′, converting all of the AMP solution into IMP solution by using adenosine deaminase enzyme,

step c3: concentrating and crystallizing the UMP solution, the GMP solution and the CMP solution obtained in step b3 respectively to obtain purified nucleotides UMP, IMP, GMP and CMP; or,

the process comprises the following steps:

step a3: in Reactor No. 1′, decomposing RNA by using nuclease P1 to obtain a mixture solution of nucleotides containing AMP, GMP, CMP and UMP,

step b3: separating the mixture solution of nucleotides obtained in step a3 by using a cation exchange resin to obtain an AMP solution, an IMF' solution, and a mixture solution containing GMP and CMIP respectively, separating the mixture solution containing GMP and CMP by using an anion exchange resin to obtain a GIMP solution and a CMP solution, and in Reactor No. 2′, converting part of the AMP solution into IMP solution by using adenosine deaminase enzyme,

preferably, in step b3, letting the mixture solution of nucleotides obtained in step a3 flow through a cation exchange resin column, collecting the effluent to obtain an UMP solution; eluting the cation exchange resin column adsorbed to reach a saturated state with deionized water, fractionally collecting the eluent to obtain a mixture solution containing GMP and CMP and an AMP solution; letting the mixture solution containing GMP and CMP flow through an anion exchange resin column, and eluting the anion exchange resin column adsorbed to reach a saturated state with deionized water, fractionally collecting the eluent to obtain a GMP solution and a CMP solution, and in Reactor No. 2′, converting part of the AMP solution into IMP solution by using adenosine deaminase enzyme to obtain a mixture solution containing AMP and IMP, letting the mixture solution containing AMP and IMP flow through a cation exchange resin column, collecting the IMP effluent, and eluting the anion exchange resin column adsorbed to reach a saturated state with deionized water, fractionally collecting the AMP eluent and the IMP eluent to obtain an AMP solution and an IMP solution,

step c3: concentrating and crystallizing the IMP solution, the AMP solution, the UMP solution, the GIMP solution and the CMP solution obtained in step b3 respectively to obtain purified nucleotides AMP, UMP, INIP, GMP and CMP;

preferably, in step b3, deaminating and converting the AMP solution to IMP solution directly; or concentrating the AMP solution prior to deaminating and converting it to IMP solution by using adenylate deaminase enzyme.

Preferably, in step a1, adding nuclease P1 and adenylate deaminase enyzme into a reactor, letting RNA react under the conditions of 30-75° C., preferably 50° C. and pH 5-6 for 3-8 hours, preferably 5 hours, and then adding activated carbon, continuing to react for 10-100 minutes, preferably 30 minutes, and then performing centrifugation, suction filtration and ultra-filtration to obtain a mixture solution of nucleotides;

preferably, in step a1, the initial concentration of RNA is 0.1-10%, preferably 1.0-6,0%; the adding amount of the nuclease P1 is 100-5000 U/g RNA, the adding amount of the adenylate deaminase enzyme is 0.1-10 U/g RNA; preferably, the adding amount of the activated carbon is 0.1-4.5% of the mass of RNA, preferably 4%.

Preferably, the steps a2 and a3 comprise: under the conditions of 20-100° C., pH 4.5-7.0, preferably 50-70° C., pH 5-6, decomposing RNA by using nuclease P1, adding activated carbon after reacting for 3-8 hours, and then continuing to react for 10-100 minutes, and then performing centrifugation, suction filtration and ultra-filtration to obtain a mixture solution of nucleotides containing AMP, GMP, CMP and UMP;

preferably, in steps a2 and a3, the initial concentration of RNA is 0.1-10%, preferably 1.0-6.0%; the adding amount of the nuclease P1 is 100-5000 U/g RNA, preferably 500-2000 U/g; preferably, the adding amount of the activated carbon is 0.1-4.5% of the mass of RNA;

preferably, in the steps b2 and b3, in the reaction of converting nucleotide AMP to nucleotide IMP by using adenylate deaminase enzyme, the adding amount of the adenylate deaminase enzyme is 0.1-10 U/g RNA, preferably 0.5-5 U/g RNA;

preferably, the reaction of converting nucleotide AMP to nucleotide IMP by using adenylate deaminase enzyme in the steps b2 and b3 is conducted under the conditions of 20-80° C., pH 4.5-7.0, more preferably 40-60° C., pH 5-6 for 1-7 hours; further preferably, adjusting the pH value by using 1-6 mol/L, preferably 1-3 mol/L, and more preferably 3 mol/L hydrochloric acid,

Preferably, the cation exchange resins are the resins in which styrene polymer or acrylic polymer is used as the skeleton and sulfonic acid group, carboxyl group or phosphoric acid group is used as the functional group; preferably, the cation exchange resins are the resins in which styrene polymer is used as the skeleton and sulfonic acid group is used as the functional group; preferably, the cation exchange resin is Amberlite IR-120, Amberlite IRC-84 or Amberlite IRA-200;

preferably, the anion exchange resins are the resins in which styrene polymer or acrylic polymer is as the skeleton, and amino group, quaternary ammonium group or tertiary amino group is as the functional group; preferably, the anion exchange resins are the resins in which styrene polymer is as the skeleton, and tertiary amino group is as the functional group; preferably, the anion exchange resin is Amberlite IRA-68, Amberlite IRA-400 or Amberlite IRA-901;

preferably, the cation exchange resin is regenerated with hydrochloride acid, and the anion exchange resin is regenerated with sodium hydroxide; preferably, the concentration of the hydrochloric acid and sodium hydroxide is 0.5-1.5 mol/L; preferably, the flow rate of the hydrochloric acid and sodium hydroxide during the resin regeneration is 0.1-1.2 BV/min;

preferably, when separating the mixture solution of nucleotides by using a cation exchange resin and an anion exchange resin, the flowing rate of the mixture solution of nucleotides through the ion exchange resin column is 0.1-3.5 BV/h, the flowing rate of elution with deionized water is 0.1-1 BV/h.

Preferably, in the process, the concentration is performed under vacuum or by using nanofiltration membrane;

preferably, the crystallization is performed after the nucleotide solution is concentrated to 100-300 g/L.

Preferably, in the process, the crystallization is performed by using ethanol-cooling method or ethanol salting-out method;

preferably, during the crystallization process, the starting temperature of the crystallization is 30-50° C. preferably 30-35° C., the feeding temperature is 8-18° C., preferably 12-15° C.

Preferably, during the production process, the crystallization is performed by using ethanol-cooling method, and during the crystallization process, ethanol is added before decreasing the temperature; or the temperature is decreased before adding ethanol; or the addition of ethanol and the decrease of temperature are performed at the same time;

preferably, the crystal growing time is 1-16 hours, preferably 5 hours during the crystallization process;

preferably, during the crystallization process, the adding rate of ethanol is 0.01-0.3 BV/h, the cooling rate is 0.5-8° C./h; preferably, the adding rate of ethanol is 0.18-0.25 BV/h for UMP, 0.15-0.25 BV/h for GMP, 0.2-0.3 BV/h for CMP, 0.15-0.32 BV/h for AMP, and 0.1-0.15 BV/h for IMP respectively; preferably, the total adding amount of the ethanol is 1.5-2.5 BV for UMP, 1.0-1.6 BV for GMP, 1.5-2.5 BV for CMP, 1.5-2.5 BV for AMP and 0.3-1.0 BV for IMP respectively.

Preferably, in the process, the crystallization is performed by using ethanol salting-out method, and during the crystallization process, solid salts or salt solutions are added into mother liquor before adding ethanol to crystallise; or ethanol is added before adding aqueous solution of salts to crystallise;

preferably, the salts are potassium salts, sodium salts or ammonium salts; more preferably, the salts are monocarboxylic salts, hydrochlorides, phosphates, sulfates, carbonates or bicarbonates; further preferably, the salt is sodium acetate, sodium phosphate or sodium sulfate;

preferably, the concentration of the aqueous solution of salts is 0.1-4.0 mol/L;

preferably, the amount of salts during the crystallization process is 0.001-0.5 g/g nucleotides.

The present invention utilizes a double-enzyme catalysis method (i.e. decomposing RNA by using nuclease P1 and converting AMP to IMP by using adenylate deaminase enzyme) to produce nucleotides, wherein the nuclease P1 is a kind of nucleic acid hydrolase, which can hydrolyze the phosphodiester bonds on RNA chains, and decompose RNA into four kinds of nucleotides UMP, GMP, CMP and AMP, the converting rate can reach nearly 85% under appropriate conditions. Adenosine deaminase enzyme can exclusively remove amino groups on AMP molecules to generate IMP, the converting rate reach above 99.9% under appropriate conditions. A double-enzyme catalysis system is established by using the above two enzymes, thus a whole biocatalytic production of five nucleotides (UMP, GMP, CMP, AMP and IMP) from RNA is realized.

Further, the present invention separates the obtained nucleotides (IMP, GMP, CMP, AMP and IMP) by employing an ion exchange resin. Under somewhat acidic conditions, UMP and IMP molecules are electrically neutral since they do not have amino groups, while GMP, CMP and AMP molecules have positive charges. When the mixture solution of these four kinds of nucleotides (UMP, GMP, CMP and AMP) or these five kinds of nucleotides (UMP, GMP, CMP, AMP and IMP) flows through a cation exchange resin column, the uncharged UMP flows out directly, while IMP is adsorbed by the resin due to the affinity of purine bases for styrene and acrylic resins, then the resin is eluted with deionized water, causing IMP, GMP, CMP and AMP be washed off from the resin successively, the separation of the above nucleotides will be realized by controlling the elution conditions.

In addition, the nucleotide is slightly soluble in ethanol, and the solubility in water decreases with the decrease of temperature; therefore, the present invention utilizes the relationship among the solubility of nucleotides in water and the temperature, as well as the changes in the amount of ethanol in the solution, and employs an ethanol-cooling method to purify the isolated nucleotides. The present invention overcomes the deficiency of the traditional single crystallization process and achieves high yield and high purity.

Compared with the existing process for producing nucleotides, the present invention at least has the following advantages:

(1) The present invention can realize a whole biocatalytic production of nucleotides and a cleaner production of five high-purity nucleotides from yeast RNA by utilizing a double-enzyme catalysis method with RNA as substrates to produce five kinds of nucleotides (UMP, GMP, CMP, AMP and IMP) having high purity, and separating and puffing the nucleotides by using an ion exchange resin separation technology and a solvent crystallization method in combination. The process is simple, environmentally-friendly and has low costs, by which the obtained product is safety and high purity, the total recovery from RNA to high-purity nucleotides can reach more than 85%, and the purity of the product can reach more than 99.98%, this method can meet the demand for nucleotides of high-purity in the infant food and pharmaceutical industries.

(2) Compared with the existing process for producing IMP, the process for producing high-purity nucleotides of the present invention simplifies the post-purification process, reduces the using amount of enzymes during catalysis and the using amount of acids and bases during the process of separation and purification, and also improves the recovery and purity of products.

### BRIEF DESCRIPTION OF THE DRAWINGS

FIG. 1 is a flowchart of the process for producing nucleotides according to example 2 of the present invention;

FIG. 2 is a flowchart of the process for producing nucleotides according to example 3 of the present invention;

FIG. 3 is a flowchart of the process for producing nucleotides according to example 4 of the present invention;

FIG. 4 is a flowchart of the process for producing nucleotides according to example 7 of the present invention.

### THE PREFERRED EMBODIMENTS OF THE PRESENT INVENTION

The present invention will be illustrated with reference to the following specific examples. The person skilled in the art will appreciate that these examples are only intended to illustrating the present invention, and should not in any way to limit the scope of the present invention.

The materials used in the examples described below, unless otherwise specified, are all from the conventional biochemical reagents store

### Example 1

### Preparation of Nuclease P1

A seed culture medium and a fermentation medium containing the following ingredients: 50 g/L glucose, 5 g/L peptone, 0.5 g/L KH₂PO₄, 0.5 g/L K₂HPO₄.3H₂O, 0.4 g/L CaCl₂ and 0.4 g/L ZnSO₄ were prepared respectively. The pH was adjusted to 6.5 before sterilization.

Preparation of nuclease P1: Penicillium citrinum N409 was inoculated in the seed culture medium prepared above, cultured at 28° C., 200 rpm for 18 hours, and then inoculated into the fermentation medium with an inoculation quantity of 10% (v/v), cultured at 28° C., 200 rpm for 24 hours, and then the fermentation broth was centrifuged at 8000 rpm, 4° C. for 15 minutes, the supernatant was taken to obtain 420 U/ml of nuclease P1 solution, which was refrigerated at 4° C. until used.

All nuclease P1 used in the following examples 2-9 was prepared according to the above method.

### Example 2

### Preparation of Nucleotides

36 g RNA of yeasts was weighed, and formulated into a 6% nucleic acid solution (in which NaOH was added to help dissolving before adjusting the pH to 5.5 with HCl), warmed to 70° C., 21000 U nuclease P1 solution preheated at 45° C. (about 50 ml) was added thereinto, and the reaction is performed for 4.5 hours at 70° C., and then 0.9 g activated carbon was added, the enzymolysis was held for 40 minutes, and then centrifugation, suction filtration and ultra-filtration were performed to obtain 610 ml mixture solution of nucleotides. After detection, the enzymolysis ratio of nucleotides was 85.54%, and the concentration of each nucleotide in the mixture solution of nucleotides was UMP 10.56 g/L, GMP 16.83 g/L, CMP 10.87g/L and AMP 13.5293 g/L.

50 g pretreated cation exchange resin Amberlite IR-120 and anion exchange resin Amberlite IRA-68 were weighed respectively and packed into columns. The mixture solution of nucleotides obtained above was loaded onto a cation exchange resin column, and flowed through the column at a rate of 3.0 BV/h, all GMP, CMP and AMP were adsorbed, and the effluent (UMP) was collected, when the cation exchange resin column reached its equilibrium adsorption capacity (the equilibrium adsorption capacity was 135 mg/g wet resin based on the total amount of GMP, CMP and AMP), the cation exchange resin column was switched. The anion exchange resin column adsorbed to reach a saturated state was eluted with deionized water, and the eluate was fractionally collected to obtain a mixture solution of GMP and CMP, and a AMP eluate, the mixture solution of GMP and CMP was flowed through the anion exchange resin column at a rate of 3.0 BV/h; when the anion exchange resin column reached its equilibrium adsorption capacity (the equilibrium adsorption capacity was 145 mg/g wet resin based on the total amount of GMP and CMP), the anion exchange resin column was switched; the anion exchange resin column absorbed to reach a saturated state was eluted with deionized water, and the GMP eluent and the CMP eluent were fractionally collected. The flowing rate of the eluents was 1 BV/h, after elution, the anion exchange resin was regenerated with 1 mol/L NaOH and the cation exchange resin was regenerated with 1 mol/L HCl, the flowing rate of NaOH and HCl during the regeneration of the resins was 0.5 BV/min.

The UMP effluent, the GMP eluent, the CMP eluent and the AMP eluent were concentrated under vacuum, which made the concentrations be promoted to 150 g/L, the AMP concentrate (about 60 ml) was transferred to the AMP reactor, 20 U (about 20 mg) adenosine deaminase powder was added thereinto, the deamination reaction was conducted at 50° C., during which 3 mol/L HCl was added to control the pH value at 5.6±0.2; after reacting for 7 hours the concentration of IMP in the enzymatic hydrolysate was 150.38 g/L, and the converting rate of AMP was 99.9999%. UMP, GMP, CMP and IMP were crystallized by using ethanol-cooling method, during the crystallization process, ethanol was added before descreasing the temperature, the adding rate of ethanol was 0.2 BV/h for UMP, 0.25 BV/h for GMP, 0.3 BV/h for CMP, 0.1 BV/h for IMP respectively, the total adding amount of ethanol was 2.0 BV for UMP, 1.6 BV for GMP, 2.5 BV for CMP and 0.8 BV for IMP respectively. The starting temperature of crystallization was 32° C., the feeding temperature was 15° C., the cooling rate was 2° C./h, and the crystal growing time was 5 hours. The crystals were filtered, recovered, and then washed with 80% cold ethanol for 2-3 times, and dried at 35° C. The purity and yield of each obtained crystal after detection were as follows: the purity of UMP was 99.985% and the yield of UMP was 98.2%; the purity of INIP was 99.992% and the yield of IMP was 99.6%; the purity of GMP was 99.987% and the yield of GMP was 99.8%; the purity of CMP was 99.984% and the yield of CMP was 99.0%.

The above operating procedure was shown in FIG. 1.

### Example 3

### Preparation of Nucleotides

50 g RNA of yeasts was weighed, and formulated into a 5% nucleic acid solution (in which NaOH was added to help dissolving before adjusting the pH to 5.5 with Ha), warmed to 50° C., and then 100000 U nuclease P1 solution preheated at 45° C. (about 240 ml) and 40 U adenosine deaminase powder (about 40 mg) were added thereinto, then the enzymatic hydrolysis-deamination reaction was performed at 50° C., during which 3 mol/L HCl was added to control the pH value at 5.5±0.2, after 5 hours, 2 g activated carbon was added, the enzymolysis was held for 30 minutes, and then centrifugation, suction filtration and ultra-filtration were performed to obtain 1020 ml mixture solution of nucleotides. After detection, the enzymolysis ratio of nucleotides was 88.56%, the converting rate of AMP was 99.9999% and the concentration of each nucleotide in the mixture solution of nucleotides was UMP 8.7692 g/L, GMP 13.9790 g/L, CMP 9.0296 g/L and IMP 11.6635 g/L.

100 g pretreated cation exchange resin Amberlite IRC-84 and anion exchange resin Amberlite IRA-400 were weighed respectively and packed into columns. The mixture solution of nucleotides obtained above was loaded onto a cation exchange resin column, and flowed through the column at a rate of 3.5 BV/h, UMP and most of IMP were not adsorbed thus flowed out directly, and the mixture solution containing UMP and IMP was collected. When the cation exchange resin column reached its equilibrium adsorption capacity (the equilibrium adsorption capacity was 125 mg/g wet resin based on the total amount of GMP, CMP and IMP), the cation exchange resin column was switched; the anion exchange resin column adsorbed to reach a saturated state was eluted with deionized water, and the IMP eluate and the mixture solution containing GMP and CMP were fractionally collected. The mixture solution of UMP and IMP and the mixture solution of GMP and CMP were flowed through the anion exchange resin column, when the anion exchange resin column reached its equilibrium adsorption capacity (the equilibrium adsorption capacity was 155 mg/g wet resin based on the total amount of UMP, IMP, GMP and CMP), the resin column was switched; the anion exchange resin column absorbed to reach a saturated state was eluted with deionized water, and the UMP eluent, the IMP eluent, the GMP eluent and CMP eluent were fractionally collected. The flowing rate of the eluents was 0.8 BV/h, after elution, the anion exchange resin was regenerated with 1.5 mol/L NaOH and the cation exchange resin was regenerated with 1.5 mon HCl, the flowing rate of NaOH and HCl during the regeneration of the resins was 1.0 BV/min.

The UMP eluent, the GMP eluent, the CMP eluent and the IMP eluent were concentrated by using nanofiltration membrane, which made the concentrations be promoted to 120 g/L, and then the crystallisation was performed by using ethanol-cooling method, during the crystallization process, ethanol was added before descreasing the temperature, the adding rate of ethanol was 0.25 BV/h for UMP, 0.2 BV/h for GMP, 0.28 BV/h for CMP, 0.15 BV/h for INIP respectively, the total adding amount of ethanol was 2.5 BV for MP, 1.5 BV for GMP, 2.0 BV for CMP and 1.0 BV for IMP respectively. The starting temperature of crystallization was 30° C., the feeding temperature was 12° C., the cooling rate was 3° C./h, the crystal growing time was 5 hours. The crystals were filtered, recovered, and then washed with 80% cold ethanol for 2-3 times, and dried at 35° C. The purity and yield of each crystal after crystallization were as follows: the purity of UMP was 99.995% and the yield of UMP was 98.91%; the purity of IMP was 99.997% and the yield of IMP was 99.72%; the purity of GMP was 99,997% and the yield of GMP was 99.63%; the purity of CMP was 99.994% and the yield of CMP was 99.38%.

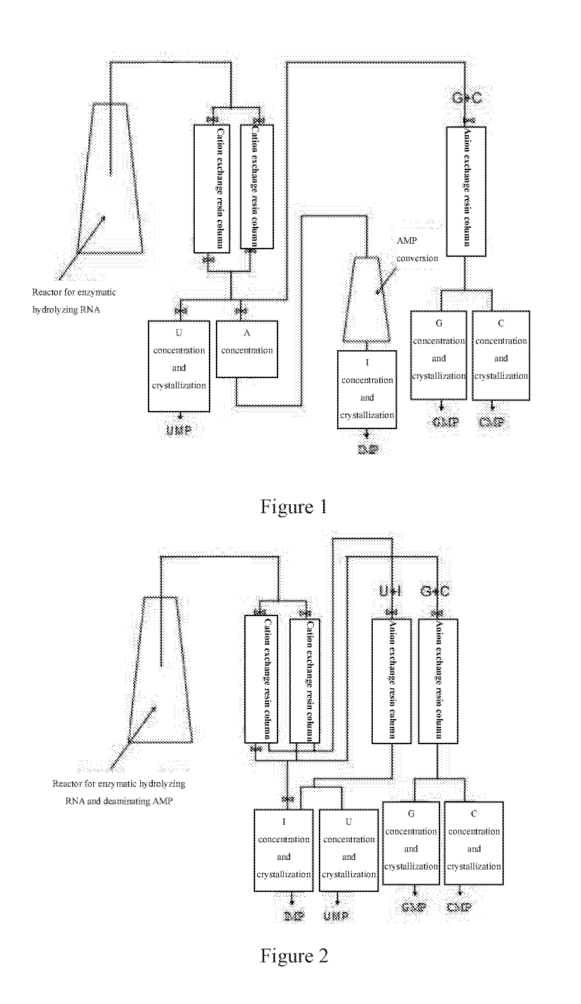

The above operating procedure was shown in FIG. 2.

### Example 4

### Preparation of Nucleotides

40 g RNA of yeasts was weighed, and formulated into a 4% nucleic acid solution (in which NaOH was added to help dissolving before adjusting the pH to 5.5 with HCl), warmed to 65° C., 25000 U nuclease P1 solution preheated at 45° C. (about 60 ml) was added thereinto, and the reaction was performed for 6 hours at 65° C., 1.2 g activated carbon was added, and the enzymolysis was held for 60 minutes, and then centrifugation, suction filtration and ultra-filtration were performed to obtain 1025 ml mixture solution of nucleotides. After detection, the enzymolysis ratio of nucleotides was 87.50%, and the concentration of each nucleotide in the mixture solution of nucleotides was UMP 6.8976 g/L, GMP 10.9951 g/L, CMP 7.1024 g/L and AMP 9.1512 g/L. The mixture solution of nucleotides was collected and transferred to a reactor, 30 U (about 30 mg) adenosine deaminase powder was added thereinto, and the deaminated conversion reaction was conducted at 50° C., during which 3 mol/L HCl was added to control the pH value at 5.6±0.2, after 6 hours, the conversion rate of AMP was 99.9999% and the concentration of IMP was 9.1772 g/L.

100 g pretreated cation exchange resin Amberlite IRA-200 and anion exchange resin Amberlite IRA-901 were weighed respectively and packed into columns. The mixture solution containing 4 kinds of nucleotides which were UMP, GMP, CMP and IMP was loaded onto the cation exchange resin column, and flowed through the column at a rate of 2.5 BV/h LIMP and most of IMP were not adsorbed thus flowed out directly, the mixture solution containing UMP and IMP was collected. When the cation exchange resin column reached its equilibrium adsorption capacity (the equilibrium adsorption capacity was 130 mg/g wet resin based on the total amount of GMP, CMP and IMP), the cation exchange resin column was switched, the anion exchange resin column adsorbed to reach a saturated state was eluted with deionized water, and the IMP eluate and the mixture solution containing GMP and CMP were fractionally collected. The mixture solution of UMP and IMP and the mixture solution of GMP and CMP were flowed through the anion exchange resin column at the rate of 2.5 BV/h, when the anion exchange resin column reached its equilibrium adsorption capacity d (the equilibrium adsorption capacity was 105 mg/g wet resin based on the total amount of UMP and IMP or the total amount of GMP and CMP), the resin column was switched, the anion exchange resin column absorbed to reach a saturated state was eluted with deionized water, the UMP eluent, the IMP eluent, the GMP eluent and CMP eluent were fractionally collected. The flowing rate of the eluents was 0.3 BV/h respectively, after elution, the anion exchange resin was regenerated with 1.2 mol/L NaOH and the cation exchange resin was regenerated with 1.2 mol/L HCl, the flowing rate of NaOH and HCl during the regeneration of the resins were 0.3 BV/min.

The UMP eluent, the GIMP eluent, the CMP eluent and the IMP eluent was concentrated by using nanofiltration membrane, which made the concentrations be promoted to 130 g/L, and then the crystallization was performed by using ethanol-cooling method, during the crystallization process, ethanol was added before descreasing the temperature, the adding rate of ethanol was 0.18 BV/h for UMP, 0.15 BV/h for GMP, 0.20 BV/h for CMP, 0.10 BV/h for IMP respectively, the total adding amount of ethanol was 2.5 BV for MP, 1.5 BV for GMP, 2.0 BV for CMP and 1.0 BV for IMP respectively. The starting temperature of crystallization was 35° C., the feeding temperature was 13° C., the cooling rate was 3.5° C./h, the crystal growing time was 5 hours. The crystals were filtered, recovered, and washed with 80% cold ethanol for 2-3 times, and dried them at 35° C. The purity and yield of each crystal after detection were as follows: the purity of UMP was 99.995% and the yield of UMP was 98.91%; the purity of IMP was 99.997% and the yield of IMP was 99.72%; the purity of GMP was 99.997% and the yield of GMP was 99.63%; the purity of CMP was 99.994% and the yield of CMP was 99.38%.

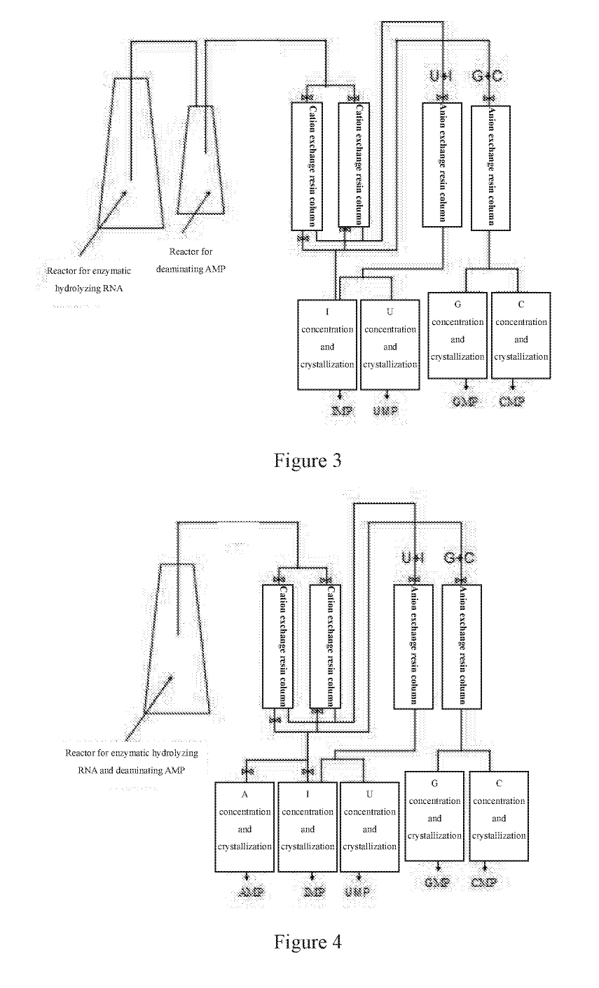

The above operating procedure was shown in FIG. 3.

### Example 5

### Preparation of Nucleotides

This example used the same method as example 2 to perform the enzymolysis of RNA, the conversion and separation of AMP by an ion exchange resin column, except that the ethanol salting-out method was adopted in this example, the crystallization process was as follows.

The UMP effluent, the GMP eluent, the CMP eluent and the AMP eluent were concentrated under vacuum, which made the concentrations be promoted to 200 g/L, then 2.0 g, 4.0 g, 3.2 g and 2.8 g sodium acetate were added thereinto respectively, and heated in water bath and stared at 25° C., ethanol was added to crystallize after the solid sodium acetate dissolved adequately, the adding rate of ethanol was 0.2 BV/h for UMP, 0.15 BV/h for GMP, 0.18 BV/h for CMP, 0.1 BV/h for IMP respectively, the addition was stopped when crystal appeared, the addition was gone on after stirring and growing crystals for 5 hours, the total adding amount of ethanol was 2.0 BV for UMP, 1.5 BV for GMP, 2.2 BV for CMP and 1.0 BV for IMP respectively. The stirring was performed for 30 min after finishing the addition, and then the solid-liquid two-phase was separated by suction filtration, the wet crystals obtained by suction filtration were washed with 85% cold ethanol for 2-3 tunes, and then dried at 35° C. The purity and yield of each crystal after detection were as follows: the purity of UMP was 99.995% and the yield of UMP was 99.2%; the purity of IMP was 99.996% and the yield of IMP was 99.7%; the purity of GMP was 99.999% and the yield of GMP was 99.8%; the purity of CMP was 99.984% and the yield of CMP was 99.3%.

### Example 6

### Preparation of Nucleotides

This example was similar to example 5, except that the time for deaminasing reaction was reduced to 4 hours, the conversion rate of AMP was 58.1614%; and this example also included the steps of separating AMP and IMP as well as crystaliszing AMP. The purity and yield of each crystal were as follows: the purity of UMP was 99.998% and the yield of UMP was 99.7%; the purity of IMP was 99.998% and the yield of IMP was 99.2%; the purity of GMP was 99.999% and the yield of GMP was 99.2%; the purity of CMP was 99.991% and the yield of CMP was 99.5%; the purity of AMP was 99.989% and the yield of AMP was 99.1%.

When part of AMP was converted, the sepration and crystallization method between AMP and IMP were as follows: 50 g pretreated cation exchange resin Amberlite IR-120 was weighed and packed into column, the mixture solution of AMP and IMP was loaded onto the cation exchange resin column, and flowed through the column at a rate of 3.0 BV/h, most of INIP were not adsorbed thus flowed out directly, and part of IMP and all AMP were adsorbed, when the cation exchange resin column reached its equilibrium adsorption capacity (the equilibrium adsorption capacity was 140 mg/g wet resin based on the total amount of AMP and IMP), the resin column was switched, the anion exchange resin column adsorbed to reach a saturated state was eluted with deionized water, the flowing rate of the eluent was 0.9 BV/h. Since the adsorption ability between IMP and the cation exchange resin was very week (only a small number of IMP were adsorbed), IMP was eluted from the resin firstly, thus achieving the separation, the IMP effluent and eluent and AMP eluent were collected, and then concentrated and crystallized. The crystallization process of AMP was as follows: the AMP eluent was concentrated under vacuum, which made the concentration be promoted to 200 g/L, 2.6 g sodium acetate was added thereinto, and heated in water bath and sttired at 25° C., ethanol was added to crystallize after the solid sodium acetate dissolved adequately, the adding rate of ethanol was 0.15 BV/h, the addition was stopped when crystal appeared, the addition was gone on after stirring and growing crystals for 5 hours the total adding amount of ethanol was 2.3 BV. The stirring was performed for 30 min after finishing the addition, and then the solid-liquid two-phase was separated by suction filtration, the wet crystals obtained by suction filtration were washed with 85% cold ethanol for 2-3 times, and then dried at 35° C.

### Example 7

### Preparation of Nucleotides

This example was similar to example 3, except that the input amount of adenosine deaminase powder was reduced to 10 U, and the conversion rate of AMP was 27.8082%, the concentration of each nucleotide in the resulting mixture solution of nucleotides was AMP 8.3961 g/L, UMP 8.7692 g/L, GMP 13.9790 g/L, CMP 9.0296 g/L, and IMP 3.2520 g/L; this example also included the steps of separating the five kinds of nucleotides which were AMP, UMP, GMP, CMP and IMP.

The method for separating the mixture of five kinds of nucleotides was as follows: 100 g pretreated cation exchange resin Amberlite IRC-84 and anion exchange resin Amberlite IRA-400 were weighed and packed into columns. The above obtained mixture solution of nucleotides was loaded onto the cation exchange resin column, and flowed through the column at a rate of 3.5 BV/h, UMP and most of IMP were not adsorbed thus flowed out directly, the mixture solution containing UMP and IMP was collected, When the cation exchange resin column reached its equilibrium adsorption capacity (the equilibrium adsorption capacity was 125 mg/g wet resin based on the total amount of AMP, GMP, CMP and IMP), the resin column was switched, the cation exchange resin column adsorbed to reach a saturated a state was eluted with deionized water, the IMP eluate, the mixture solution containing GMP and CMP, and AMP eluate were fractionally collected. The mixture solution of UMP and IMP and the mixture solution of GMP and CMP were flowed through the anion exchange resin column, when the anion exchange resin column reached its equilibrium adsorption capacity (the equilibrium adsorption capacity was 155 mg/g wet resin based on the total amount of UMP, IMP, GMP and CMP), the resin column was switched, the anion exchange resin column adsorbed to reach a saturated state was eluted with deionized water, the UMP eluent, the IMP eluent, the GMP eluent and CMP eluent were fractionally collected. The flowing rate of the eluents was 0.8 BV/h respectively, after elution, the anion exchange resin was regenerated with 1.5 mon NaOH and the cation exchange resin was regenerated with 1.5 mol/L HCl, the flow rate of NaOH and HCl during the resins regeneration process was 0.9 BV/min.

The UMP eluent, the GMP eluent, the CMP eluent, the AMP eluent and the IMP eluent were concentrated by using nanofiltration membrane, which made the concentrations be promoted to 120 g/L, and then the crystallisation was performed by using ethanol-cooling method, during the crystallization process, ethanol was added before descreasing the temperature, the adding rate of ethanol was 0.25 BV/h for UMP, 0.2 BV/h for GMP, 0.28 BV/h for CMP, 0.18 BV/h for AMP and 0.15 BV/h for IMP respectively, the total adding amount of ethanol was 2.5 BV for UMP, 1.5 BV for GMP, 2.0 BV for CMP, 2.2 BV for AMP and 1.0 BV for IMP respectively. The starting temperature of crystallization was 30° C., the feeding temperature was 12° C., the cooling rate was 3.0° C./h, the crystal growing time was 5 hours. The crystals were recovered by suction filtration, and washed with 80% cold ethanol for 2-3 times, and then dried at 35° C. The purity and yield of each resulting crystal after detection were as follows: the purity of UMP was 99.999% and the yield of UMP was 99.8%; the purity of IMP was 99.999% and the yield of IMP was 99.8%; the purity of GMP was 99.997% and the yield of GMP was 99.5%; the purity of CMP was 99.993% and the yield of CMP was 99.7%; the purity of AMP was 99.991% and the yield of AMP was 99.6%

### Example 8

### Preparation of Nucleotides

This example was similar to example 5, except that sodium phosphate was used as the salt when performing the crystallisation by using ethanol salting-out method. The purity and yield of each crystal were as follows: the purity of UMP was 99.990% and the yield of UMP was 99.3%; the purity of IMP was 99.995% and the yield of IMP was 99.8%; the purity of GMP was 99.991% and the yield of GMP was 99.9%; the purity of CMP was 99.982% and the yield of CMP was 99.7%.

### Example 9

### Preparation of Nucleotides

This example was similar to example 8, except that sodium sulfate was used as the salt when performing the crystallisation by using ethanol salting-out method. The purity and yield of each crystal were as follows: the purity of UMP was 99.996% and the yield of UMP was 99.7%; the purity of IMP was 99.998% and the yield of IMP was 99.9%; the purity of GMP was 99.992% and the yield of GMP was 99.8%; the purity of CMP was 99.989% and the yield of CMP was 99.9%.

### Example 10

### Preparation of Nucleotides

This example was similar to example 2, except that when performing the crystallisation by using ethanol-cooling method, the temperature was descreased before adding ethanol. The purity and yield of each crystal were as follows: the purity of UMP was 99.998% and the yield of UMP was 98.5%; the purity of IMP was 99.994% and the yield of IMP was 99.7%; the purity of GMP was 99.985% and the yield of GMP was 99,6%; the purity of CMP was 99.989% and the yield of CMP was 99.4%.

### Example 11

### Preparation of Nucleotides

This example was similar to example 5, except that when performing the crystallisation by using ethanol salting-out method, ethanol was added before adding 2 mol/L sodium acetate solution, but the total adding amount of ethanol. and sodium acetate solution remained constant. The purity and yield of each crystal were as follows: the purity of UMP was 99.994% and the yield of UMP was 99.3%; the purity of IMP was 99.997% and the yield of IMP was 99.6%; the purity of GMP was 99.979% and the yield of GMP was 99.9%; the purity of CMP was 99.994% and the yield of CMP was 99.2%.

### Example 12

### Preparation of Nucleotides

This example was similar to example 2, except that when performing the crystallisation by using ethanol-cooling method, the descrease of the temperature and the addition of ethanol were performed simultaneously. The purity and yield of each crystal were as follows: the purity of UMP was 99.968% and the yield of UMP was 98.8%; the purity of IMP was 99.974% and the yield of IMP was 99.9%; the purity of GMP was 99.965% and the yield of GMP was 99.7%; the purity of CMP was 99.959% and the yield of CMP was 99.6%.

### Example 13

### Preparation of Nucleotides

This example was similar to example 11, except that when performing the crystallisation by using ethanol salting-out method the conceration of sodium. acetate was 3 mol/L, but the total amount of sodium acetate and ethanol remained constant.

### Example 14

### Preparation of Nucleotides

This example was similar to example 13, except that when performing the crystallisation by using ethanol salting-out method the used salt was sodium sulfate.

1. A process for the production of nucleotides comprising:
decomposing RNA by using nuclease P1 to obtain nucleotides AMP, GMP, CMP and UMP, converting part or all of the nucleotide AMP into nucleotide IMP by using adenosine deaminase enzyme, separating the obtained nucleotides by using an ion exchange resin, and then concentrating and crystallizing to obtain purified nucleotides AMP, GMP, CMP, UMP and IMP or purified nucleotides GMP, CMP, UMP and IMP.

2. The process according to claim 1, wherein the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in the same or different reactors.

3. The process according to claim 2, wherein the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in the same reactor, the process comprises the following steps:
step a1: in a reactor, decomposing RNA by using nuclease P1 to obtain nucleotides AMP, GMP, CMP and UMP, converting part or all of the nucleotide AMP into nucleotide IMP by using adenosine deaminase enzyme to obtain a mixture solution of nucleotides comprising AMP, GMP, CMP, IMP and UMP or a mixture solution of nucleotides comprising GMP, CMP, IMP and UMP,
step b1: separating the mixture solution of nucleotides obtained in step a1 by using an ion exchange resin, and then oncentrating and crystallizing to obtain purified nucleotides AMP, GMP, CMP, UMP and IMP or obtain purified nucleotides GMP, CMP, UMP and IMP;
preferably, the process comprises the following steps:
step a1: adding nuclease P1 and adenosine deaminase enzyme into a reactor, decomposing RNA to obtain nucleotides AMP, GMP, CMP and UMP, converting all of the nucleotide AMP into nucleotide IMP to obtain a mixture solution of nucleotides comprising GMP, CMP, IMP and UMP,
step b1: separating the mixture solution of nucleotides obtained in step a1 by using an ion exchange resin to obtain an IMP solution, a mixture solution containing UMP and IMP, as well as a mixture solution containing GMP and CMP respectively, separating the mixture solution containing UMP and IMP, as well as the mixture solution containing GMP and CMP respectively by using an anion exchange resin to obtain an UMP solution, an IMP solution, a GMP solution and a CMP solution, and then concentrating and crystallizing the UMP solution, the IMP solution, the GMP solution and the CMP solution respectively to obtain purified nucleotides UMP, IMP, GMP and CMP,
preferably, in step b1, letting the mixture solution of nucleotides obtained in step a1 flow through a cation exchange resin column, collecting the effluent to obtain a mixture solution containing UMP and IMP; eluting the cation exchange resin column adsorbed to reach a saturated state with deionized water, fractionally collecting the eluent to obtain an IMP solution and a mixture solution containing GMP and CMP; letting the mixture solution containing GMP and CMP as well as the mixture solution containing UMP and IMP flow through an anion exchange resin column, and eluting the anion exchange resin column adsorbed to reach a saturated state with deionized water, fractionally collecting the eluent to obtain an UMP solution, an IMP solution, a GMP solution and a CMP solution, and then concentrating and crystallizing the UMP solution, the IMP solution, the GMP solution and the CMP solution respectively to obtain purified nucleotides UMP, IMP, GMP and CMP;
preferably, the process comprises the following steps:
step a1: adding nuclease P1 and adenosine deaminase enzyme into a reactor, decomposing RNA to obtain nucleotides AMP, GMP, CMP and UMP, converting part of the nucleotide AMP into nucleotide IMP to obtain a mixture solution of nucleotides comprising AMP, GMP, CMP, IMP and UMP,
step b1: separating the mixture solution of nucleotides obtained in step a1 by using a cation exchange resin to obtain an AMP solution, an IMP solution, a mixture solution containing UMP and IMP and a mixture solution containing GMP and CMP respectively, separating the mixture solution of nucleotides containing UMP and IMP, as well as the mixture solution containing GMP and CMP respectively by using an anion exchange resin to obtain an UMP solution, an IMP solution, a GMP solution and a CMP solution, and then concentrating and crystallizing the AMP solution, the UMP solution, the IMP solution, the GMP solution and the CMP solution respectively to obtain purified nucleotides AMP, UMP, IMP, GMP and CMP,
preferably, in step b1, letting the mixture solution of nucleotides obtained in step a1 flow through a cation exchange resin column, collecting the effluent to obtain a mixture solution containing UMP and IMP; eluting the cation exchange resin column adsorbed to reach a saturated state with deionized water, fractionally collecting the eluent to obtain an AMP solution, an IMP solution and a mixture solution containing GMP and CMP; letting the mixture solution containing GMP and CMP and the mixture solution containing UMP and IMP flow through an anion exchange resin column, and eluting the anion exchange resin column adsorbed to reach a saturated state with deionized water, fractionally collecting the eluent to obtain an UMP solution, an IMP solution, a GMP solution and a CMP solution, and then concentrating and crystallizing the AMP solution, the UMP solution, the IMP solution, the GMP solution and the CMP solution to obtain purified nucleotides AMP, UMP, IMP, GMP and CMP.

4. The process according to claim 2, wherein the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in different reactors, the process comprises the following steps:
step a2: in Reactor No. 1, decomposing RNA by using nuclease P1 to obtain a mixture solution of nucleotides containing AMP, GMP, CMP and UMP,
step b2: transferring the mixture solution of nucleotides obtained in the step a2 to Reactor No. 2, converting AMP in the mixture solution of nucleotides into IMP by using adenosine deaminase enzyme to obtain a mixture solution of nucleotides containing IMP,
step c2: separating the mixture solution of nucleotides containing IMP obtained in step b2 by using an ion exchange resin, and then concentrating and crystallizing to obtain purified nucleotide;
preferably, the process comprises the following steps:
step a2: in Reactor No. 1, decomposing RNA by using nuclease P1 to obtain a mixture solution of nucleotides containing AMP, GMP, CMP and UMP,
Step b2: transferring the mixture solution of nucleotides obtained in the step a2 to Reactor No. 2, converting all of AMP in the mixture solution of nucleotides into IMP by using adenosine deaminase enzyme to obtain a mixture solution of nucleotides containing IMP,
step c2: separating the mixture solution of nucleotides containing IMP obtained in step b2 by using a cation exchange resin to obtain an IMP solution, a mixture solution containing UMP and IMP, as well as a mixture solution containing GMP and CMP respectively, separating the mixture solution containing UMP and IMP as well as the mixture solution containing GMP and CMP respectively by using an anion exchange resin to obtain an UMP solution, an IMP solution, a GMP solution and a CMP solution, and then concentrating and crystallizing the UMP solution, the IMP solution, the GMP solution and the CMP solution to obtain purified nucleotides UMP, IMP, GMP and CMP,
preferably, in step c2, letting the mixture solution of nucleotides containing IMP obtained in step b2 flow through the cation exchange resin column, collecting the effluent to obtain a mixture solution containing UMP and IMP; eluting the cation exchange resin column adsorbed to reach a saturated state with deionized water, fractionally collecting the eluent to obtain an IMP solution, and a mixture solution containing GMP and CMP; letting the mixture solution containing GMP and CMP and the mixture solution containing UMP and IMP flow through the anion exchange resin column respectively, and eluting the anion exchange resin column adsorbed to reach a saturated state with deionized water, fractionally collecting the eluent to obtain an UMP solution, an IMP solution, a GMP solution and a CMP solution, and then concentrating and crystallizing the UMP solution, the IMP solution, the GMP solution and the CMP solution respectively to obtain purified nucleotides UMP, IMP, GMP and CMP.

5. The process according to claim 2, wherein the reaction of decomposing RNA by using nuclease P1 to obtain nucleotides AMP, GMP, CMP and UMP and the reaction of converting part or all of the nucleotide AMP into nucleotide IMP by using adenosine deaminase enzyme are carried out in different reactors, the process comprises the following steps:
step a3: in Reactor No. 1′, decomposing RNA by using nuclease P1 to obtain a mixture solution of nucleotides containing AMP, GMP, CMP and UMP,
step b3: separating the mixture solution of nucleotides obtained in step a3 by using a cation exchange resin to obtain an AMP solution, an UMP solution, and a mixture solution containing GMP and CMP respectively, separating the mixture solution containing GMP and CMP by using an anion exchange resin to obtain a GMP solution and a CMP solution, and in Reactor No. 2′, converting all of the AMP solution into IMP solution by using adenosine deaminase enzyme,
preferably, in step b3, letting the mixture solution of nucleotides obtained in step a3 flow through a cation exchange resin column, collecting the effluent to obtain an UMP solution; eluting the cation exchange resin column adsorbed to reach a saturated state with deionized water, fractionally collecting the eluent to obtain a mixture solution containing GMP and CMP and an AMP solution; letting the mixture solution containing GMP and CMP flow through an anion exchange resin column, and eluting the anion exchange resin column adsorbed to reach a saturated state with deionized water, fractionally collecting the eluent to obtain a GMP solution and a CMP solution, and in Reactor No. 2′, converting all of the AMP solution into IMP solution by using adenosine deaminase enzyme,
step c3: concentrating and crystallizing the UMP solution, the GMP solution and the CMP solution obtained in step b3 respectively to obtain purified nucleotides UMP, IMP, GMP and CMP; or,
the process comprises the following steps:
step a3: in Reactor No. 1′, decomposing RNA by using nuclease P1 to obtain a mixture solution of nucleotides containing AMP, GMP, CMP and UMP,
step b3: separating the mixture solution of nucleotides obtained in step a3 by using a cation exchange resin to obtain an AMP solution, an UMP solution, and a mixture solution containing GMP and CMP respectively, separating the mixture solution containing GMP and CMP by using an anion exchange resin to obtain a GMP solution and a CMP solution, and in Reactor No. 2′, converting part of the AMP solution into IMP solution by using adenosine deaminase enzyme,
preferably, in step b3, letting the mixture solution of nucleotides obtained in step a3 flow through a cation exchange resin column, collecting the effluent to obtain an UMP solution; eluting the cation exchange resin column adsorbed to reach a saturated state with deionized water, fractionally collecting the eluent to obtain a mixture solution containing GMP and CMP and the AMP solution; letting the mixture solution containing GMP and CMP flow through the anion exchange resin column, and eluting the anion exchange resin column adsorbed to reach a saturated state with deionized water, fractionally collecting the eluent to obtain a GMP solution and a CMP solution, and in Reactor No. 2′, converting part of the AMP solution into IMP solution by using adenosine deaminase enzyme to obtain a mixture solution containing AMP and IMP, letting the mixture solution containing AMP and IMP flow through a cation exchange resin column, collecting the IMP effluent, and eluting the anion exchange resin column adsorbed to reach a saturated state with deionized water, fractionally collecting the AMP eluent and the IMP eluent to obtain an AMP solution and an IMP solution,
step c3: concentrating and crystallizing the IMP solution, the AMP solution, the UMP solution, the GMP solution and the CMP solution obtained in step b3 respectively to obtain purified nucleotides AMP, UMP, IMP, GMP and CMP;
preferably, in step b3, deaminating and converting the AMP solution to IMP solution directly; or, concentrating the AMP solution prior to deaminating and converting it to IMP solution by using adenylate deaminase enzyme.

6. The process according to claim 3, wherein in step a1, adding nuclease P1 and adenylate deaminase enzyme into a reactor, letting RNA react under the conditions of 30-75° C., preferably 50° C. and pH 5-6 for 3-8 hours, preferably 5 hours, and then adding activated carbon, continuing to react for 10-100 minutes preferably 30 minutes, and then performing centrifugation, suction filtration and ultra-filtration to obtain a mixture solution of nucleotides;
preferably, in step a1, the initial concentration of RNA is 0.1-10%, preferably 1.0-6.0%; the adding amount of the nuclease P1 is 100-5000 U/g RNA, the adding amount of the adenylate deaminase enzyme is 0.1-10 U/g RNA; preferably, the adding amount of the activated carbon is 0.1-4.5% of the mass of RNA, preferably 4%.

7. The process according to claim 4, wherein the steps a2 and a3 comprise: under the conditions of 20-100° C., pH 4.5-7.0, preferably 50-70° C., pH 5-6, decomposing RNA by using nuclease P1, adding activated carbon after reacting for 3-8 hours, and then continuing to react for 10-100 minutes, and then performing centrifugation, suction filtration and ultra-filtration to obtain a mixture solution of nucleotides containing AMP, GMP, CMP and UMP;
preferably, in steps a2 and a3, the initial concentration of RNA is 0.1-10%, preferably 1.0-6.0%; the adding amount of the nuclease P1 is 100-5000U/g RNA, preferably 500-2000 U/g; preferably, the adding amount of the activated carbon is 0.1-4.5% of the mass of RNA;
preferably, in steps b2 and b3, in the reaction of converting nucleotide AMP to nucleotide IMP by using adenylate deaminase enzyme, the adding amount of the adenylate deaminase enzyme is 0.1-10 U/g RNA, Preferably 0.5-5 U/g RNA;
preferably, the reaction of converting nucleotide AMP to nucleotide IMP by using adenylate deaminase enzyme in steps b2 and b3 is conducted under the conditions of 20-80° C., pH 4.5-7.0, more preferably 40-60° C., pH 5-6 for 1-7 hours;
further preferably, adjusting the pH value by using 1-6 mol/L, preferably 1-3 mol/L, and more preferably 3 mol/L hydrochloric acid.

8. The process according to claim 3, wherein the cation exchange resins are the resins in which styrene polymer or acrylic polymer is used as the skeleton and sulfonic acid group, carboxyl group or phosphoric acid group is used as the functional group; preferably, the cation exchange resins are the resins in which styrene polymer is used as the skeleton and sulfonic acid group is used as the functional group; preferably, the cation exchange resin is Amberlite IR-120, Amberlite IRC-84 or Amberlite IRA-200;
preferably, the anion exchange resins are the resins in which styrene polymer or acrylic polymer is as the skeleton, and amino group, quaternary ammonium group or tertiary amino group is as the functional group; preferably, the anion exchange resins are the resins in which styrene polymer is as the skeleton, and tertiary amino group is as the functional group; preferably, the anion exchange resin is Amberlite IRA-68, Amberlite IRA-400 or Amberlite IRA-901;
preferably, the cation exchange resin is regenerated with hydrochloride acid, and the anion exchange resin is regenerated with sodium hydroxide; preferably, the concentration of the hydrochloric acid and sodium hydroxide is 0.5-1.5 mol/L; preferably, the flow rate of the hydrochloric acid and sodium hydroxide during the resin regeneration is 0.1-1.2 BV/min;
preferably, when separating the mixture solution of nucleotides by using a cation exchange resin and an anion exchange resin, the flowing rate of the mixture solution of nucleotides through the ion exchange resin column is 0.1-3.5 BV/h, the flowing rate of elution with deionized water is 0.1-1 BV/h.

9. The process according to claim 1, wherein in the process, the concentration is performed under vacuum or by using nanofiltration membrane;
preferably, the crystallization is performed after the nucleotide solution is concentrated to 100-300 g/L.

10. The process according to claim 1, wherein in the process, the crystallization is performed by using ethanol-cooling method or ethanol salting-out method;
preferably, during the crystallization process, the starting temperature of the crystallization is 30-50° C., preferably 30-35° C., the feeding temperature is 8-18° C., preferably 12-15° C.

11. The process according to claim 10, wherein in the process, the crystallization is performed by using ethanol-cooling method, and during the crystallization process, ethanol is added before decreasing the temperature; or the temperature is decreased before adding ethanol; or the addition of ethanol and the decrease of temperature are performed at the same time;
preferably, the crystal growing time is 1-16 hours, preferably 5 hours; during the crystallization process
preferably, during the crystallization process, the adding rate of ethanol is 0.01-0.3 BV/h, the cooling rate is 0.5-8° C./h; preferably, the adding rate of ethanol is 0.18-0.25 BV/h for UMP, 0.15-0.25 BV/h for GMP, 0.2-0.3 BV/h for CMP, 0.15-0.32 BV/h for AMP, and 0.1-0.15 BV/h for IMP respectively; preferably, the total adding amount of ethanol is 1.5-2.5 BV for UMP, 1.0-1.6 BV for GMP, 1.5-2.5 BV for CMP, 1.5-2.5 BV for AMP and 0.3-1.0 BV for IMP respectively.

12. The process according to claim 10, wherein, in the process, the crystallization is performed by using ethanol salting-out method, and during the crystallization process, solid salts or salt solutions are added into mother liquor before adding ethanol to crystallise; or ethanol is added before adding aqueous solution of salts to crystallise;
preferably, the salts are potassium salts, sodium salts or ammonium salts;
more preferably, the salts are monocarboxylic salts, hydrochlorides, phosphates, sulfates, carbonates or bicarbonates; further preferably, the salt is sodium acetate, sodium phosphate or sodium sulfate;
preferably, the concentration of the aqueous solution of salts is 0.1-4.0 mol/L;
preferably, the amount of salts during the crystallization process is 0.001-0.5 g/g nucleotides.

13. The process according to claim 5, wherein the steps a2 and a3 comprise: under the conditions of 20-100° C., pH 4.5-7.0, preferably 50-70° C., pH 5-6, decomposing RNA by using nuclease P1, adding activated carbon after reacting for 3-8 hours, and then continuing to react for 10-100 minutes, and then performing centrifugation, suction filtration and ultra-filtration to obtain a mixture solution of nucleotides containing AMP, GMP, CMP and UMP;
preferably, in steps a2 and a3, the initial concentration of RNA is 0.1-10%, preferably 1.0-6.0%; the adding amount of the nuclease P1 is 100-5000 U/g RNA, preferably 500-2000 U/g; preferably, the adding amount of the activated carbon is 0.1-4.5% of the mass of RNA;
preferably, in steps b2 and b3, in the reaction of converting nucleotide AMP to nucleotide IMP by using adenylate deaminase enzyme, the adding amount of the adenylate deaminase enzyme is 0.1-10 U/g RNA, Preferably 0.5-5 U/g RNA;
preferably, the reaction of converting nucleotide AMP to nucleotide IMP by using adenylate deaminase enzyme in steps b2 and b3 is conducted under the conditions of 20-80° C., pH 4.5-7.0, more preferably 40-60° C., pH 5-6 for 1-7 hours;
further preferably, adjusting the pH value by using 1-6 mol/L, preferably 1-3mol/L, and more preferably 3 mol/L hydrochloric acid.
